(19) United States
(12) Patent Application Publication
(10) Pub. No.: US 2026/0004907 A1
(43) Pub. Date: Jan. 1, 2026

(54) COMPUTER-IMPLEMENTED SYSTEMS AND METHODS FOR AUTOMATING PATIENT ANALYSIS AND PRESCRIPTION GENERATION

(71) Applicant: Tod Stillson, Plymouth, IN (US)

(72) Inventor: Tod Stillson, Plymouth, IN (US)

(21) Appl. No.: 18/756,093

(22) Filed: Jun. 27, 2024

(51) Int. Cl. G16H 20/10; G06Q 30/0601; G16H 10/60; G16H 50/20

(52) U.S. Cl. CPC G16H 20/10; G06Q 30/0639; G16H 10/60; G16H 50/20

(57) ABSTRACT

A system for automating patient condition analysis and prescription generation is disclosed, including an application server in operable communication with the user network to host an application program for providing healthcare services and enabling an interaction between a healthcare provider and a patient. A conversation engine analyzes and evaluates patient symptoms and patient medical information to identify a non-qualified condition, a qualified condition that allows for a prescription, and a high risk condition that necessitates a face-to-face medical evaluation. A comparator is in communication with the conversation engine to compare the patient symptoms and patient medical information to a clinical dataset to enable the determination of a medication from a medication dataset. A prescription generation module associates the medication with the patient to generate an e-prescription which is transmitted to a pharmacy.

### TECHNICAL FIELD

The embodiments disclosed herein generally relate to computerized systems and methods for automating healthcare services.

### BACKGROUND

Healthcare services include various aspects of patient intake, management, and care which has historically been provided in a face-to-face interaction between a healthcare provider and patient. In recent years, telehealth has become increasingly prominent as advances in computer technologies have enabled healthcare providers to provide healthcare services using a network environment (i.e., the Internet). These advances have resulted in the increased access to healthcare services and provide a benefit to the healthcare system and patients alike.

While many patients require an in-person consultation and examination, some may benefit from the implementation of telehealth capabilities, thus forgoing the in-person doctors visit to receive the care they need. While telehealth system can eliminate the need for an in-person consultation, current telehealth platforms often require a remote consultation using chat and video communications which results time being spent by the healthcare provider (i.e., a physician, nurse, etc.) when communicating and assessing the patient.

### SUMMARY OF THE INVENTION

This summary is provided to introduce a variety of concepts in a simplified form that is further disclosed in the detailed description of the embodiments. This summary is not intended for determining the scope of the claimed subject matter.

The embodiments provided herein relate to a system for automating patient condition analysis and prescription generation, including an application server in operable communication with the user network to host an application program for providing healthcare services and enabling an interaction between a healthcare provider using artificial intelligence and a patient. The application program includes a user interface module for providing access to the application program via the user computing device. A conversation engine analyzes and evaluates patient symptoms and patient medical information to identify a non-qualified condition, a qualified condition that allows for a prescription, and a high risk condition that necessitates a face-to-face medical evaluation. A comparator is in communication with the conversation engine to compare the patient symptoms and patient medical information to a clinical dataset to enable the determination of a medication from a medication dataset. A prescription generation module associates the medication with the patient to generate an e-prescription which is transmitted to a pharmacy.

The embodiments provide consumers with a means of procuring a medication without an in-person visit to a medical professional. This is beneficial for individuals who are hesitant to interact face-to-face with a medical professional, who do not have easy access to medical care, or unable to travel to visit a medical professional. The system provides an immediate assessment of a condition, efficient treatment planning, and saves time and resources. Medical professionals benefit by receiving an efficient means of providing care to patients, without requiring the time-consuming task of scheduling and consulting with the patient in-person. Insurance providers benefit from the system by receiving a service which streamlines the prescription procurement processes.

The embodiments include an AI-enabled system which automates various processes related to the assessment of patient conditions, determination of a suitable medication which is effective in treating the condition and provides an efficient means of generating and delivering a prescription to a pharmacy. The AI-enabled system is capable of automatically analyzing symptoms, medical histories, conditions, complications, etc. to determine a suitable treatment for the patient. Further, the system may be able to prevent, or at least reduce the misuse of antibiotics using the AI engine.

In one aspect, the system includes a location module to determine the location of the patient and to provide a list of pharmacies within a predefined radius of the location of the patient.

In one aspect, the system includes a payment processing module to receive a payment from the patient and to distribute at least a portion of the payment to one or more recipients.

In one aspect, the payment processing module is operable to generate an invoice associated with the e-prescription.

In one aspect, a patient database stores a plurality of patient information comprising: the plurality of patient symptoms, a patient medical history, and a plurality of patient identifying information.

In one aspect, a medication database stores the medication dataset, wherein the medication dataset is comprised of a plurality of antibiotics.

In one aspect, the medication database stores a plurality of medications effective in treating one or more of a plurality of acute conditions comprising the clinical dataset.

In one aspect, the conversation engine includes an AI-enabled chat bot operable to perform the following: transmit automated communications to the patient, receive a plurality of patient-input communications, and analyze the plurality of patient-input communication to recommend the medication to the patient to identify a patient condition and determine if it is a qualified condition for a prescription and then recommend the medication to the patient based on the product algorithm.

In one aspect, an administrative portal is accessible via the healthcare provider. The administrative portal is operable to permit the healthcare provider to review and to validate the e-prescription.

A method for automating patient analysis and prescription generation is disclosed. A conversation engine analyzes a plurality of patient symptoms and a plurality of patient medical information to evaluate the plurality of patient symptoms and the plurality of patient medical information. The conversation engine then compares, via a comparator, the plurality of patient symptoms and the plurality of patient medical information to a clinical dataset to enable the determination of a medication from a medication dataset. A prescription generation module associates the medication with the patient to generate an e-prescription. A healthcare provide accessing the administrator portal may then validate the e-prescription after it has been transmitted to the pharmacy by the AI-enabled bot.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of a computer system architecture, according to some embodiments;

FIG. 2 illustrates a block diagram of an application program in operable communication with the computer system, according to some embodiments;

FIG. 3 illustrates a block diagram of the databases and application program, according to some embodiments;

FIG. 4A illustrates a flowchart of a process for automating patient condition analysis and prescription generation, according to some embodiments;

FIG. 4B illustrates a flowchart of a process for automating patient condition analysis and prescription generation, according to some embodiment; and

FIG. 4C illustrates a flowchart of a process for automating patient condition analysis and prescription generation, according to some embodiment.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to systems and methods for automating patient condition analysis and prescription generation. The system provides an AI-enabled direct-to-consumer health platform designed to virtually treat various common medical conditions. The system combines medical expertise with cutting-edge technology to deliver personalized treatment. The platform's algorithm analyzes symptoms and medical history to provide accurate auto-delivered prescriptions from a licensed physician, separating it from traditional telehealth methods.

In some embodiments, the system may integrate physician oversight with AI and ML systems through the use of a chat bot which autonomously interacts with patients. The system may offer a free assessment to solve a common consumer question of “Do I Need An Antibiotic?” The AI-powered assessment will support the CDC antibiotic stewardship initiative to reduce the over-prescribing of antibiotics. However, the assessment will help determine a consumer's qualification for online treatment of various common conditions.

In some embodiments, the system uses an AI algorithm to analyze a patient's symptoms and medical history, leading to accurate and efficient diagnoses of a qualified condition. Non-protocol conditions and critical conditions will be referred to face-to-face medical evaluation. Based on the AI's analysis, personalized treatment plans are created, including auto-delivered prescriptions that are sorted to the least expensive option based on the consumer's zip code (or other parameters as discussed herein).

In some embodiments, the system may be operable to determine if the patient's condition is treatable through the use of antimicrobials. If the user's condition is not treatable by antimicrobials, measures may be taken to direct the patient to sufficient care. If a prescription for an antimicrobials is appropriate and a prescription is selected by the patient, the system will auto-prescribe the medication on behalf of the licensed physician for pickup or delivery.

A licensed physician may then review the AI's & consumer-powered process of condition assessment and treatment plans. This is an oversite review for quality, precision, and safety analysis. The prescriptions will be auto-sent by AI and the physician review process (i.e., signing off) will occur asynchronously.

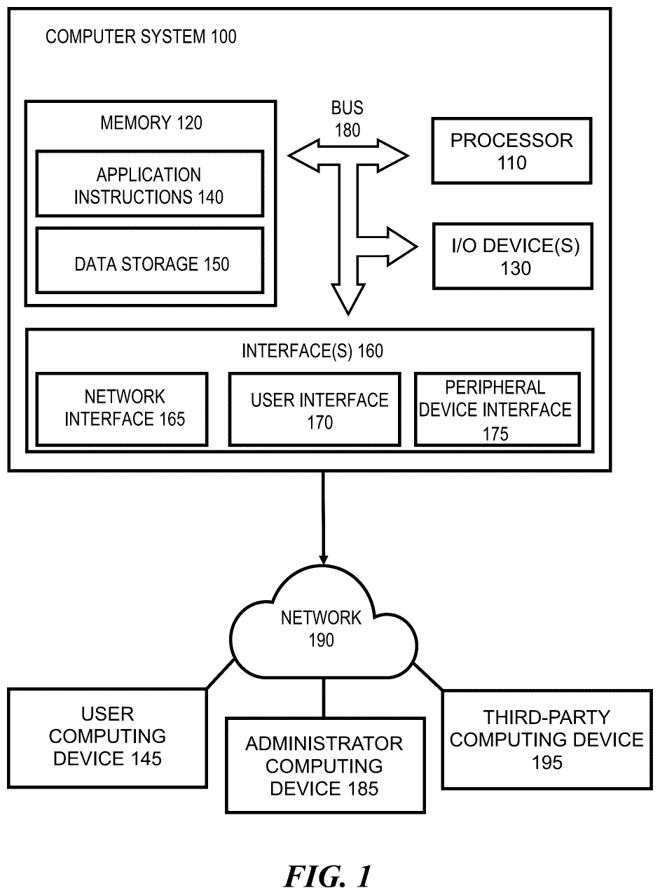

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., Android, C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a user computing device 145, an administrator computing device 185 and a third-party computing device 195 each in communication via the network 190. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

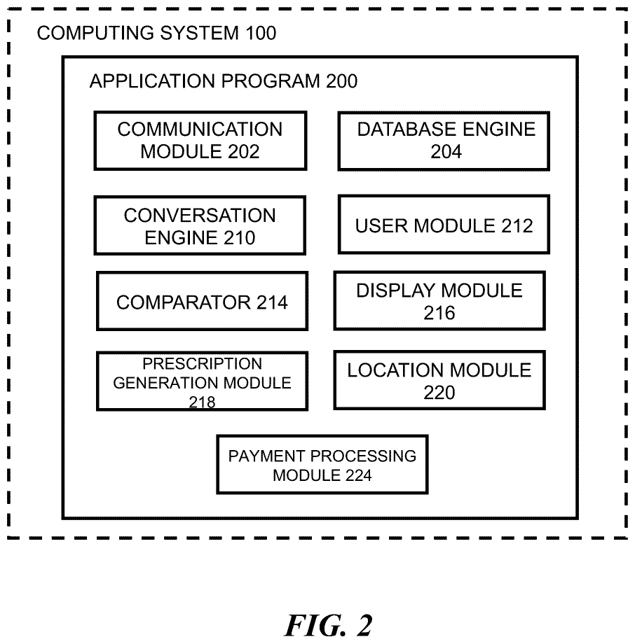

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, a conversation engine 210, a user module 212, a comparator module 214, a display module 216, a prescription generation module 218, a location module 220, and a payment processing module 224.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195.

In some embodiments, the communication module 202 may be used to enable communications between any combination of patients, pharmacies, medical professionals, AI bots, administrators, and other users of the system, including computer-implemented systems. The communication module 202 may be utilized to transmit medical information associated with patients, medication information, pharmacy information, medical professional information, etc. Further, the communication module 202 may be used to transmit an e-prescription to a pharmacy.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

In some embodiments, the database engine 204 may be operable to transmit and receive information related to patients, medical professionals, medications, pharmacies, and the like.

In some embodiments, the conversation engine 210 is operable to analyze a plurality of patient symptoms and medical information received from patient inputs into the user interface. In such, the conversation engine 210 may evaluate the patient symptoms and medical information. The conversation engine 210 is in operable communication with the comparator 214 to compare the patient symptoms and patient medical information to a clinical dataset to enable the determination of a suitable medication from the medical dataset.

In some embodiments, the user module 212 facilitates the creation of a user account for the application system. The user account may permit the user to input user information, product user credentials, preferences, etc.

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored.

In some embodiments, the prescription generation module 218 associates the medication with the patient which enables the generation of an e-prescription. This e-prescription is then transmitted, via the communication module 202, to the pharmacy which is tasked with filling the prescription.

In some embodiments, the prescription generation module 218 may enable the patient to select from a listing of pharmacies in order to select a specific pharmacy that may fill the prescription. This allows the user to select a pharmacy which is most convenient for them.

In some embodiments, the location module 220 is operable to determine the location of the patient to provide the list of pharmacies within a predefined radius of the location of the patient. The location module 220 may also be operable to determine the location of the patient for the purposes of verifying the patient's current location for regulatory compliance.

In some embodiments, the payment processing module 224 is operable to process a payment between the patient, the medical professional, the pharmacy, an insurance provider, and/or the system itself. The payment processing module 224 may distribute funds received from the patient to any combination of recipients. An invoice may be generated by the payment processing module 224.

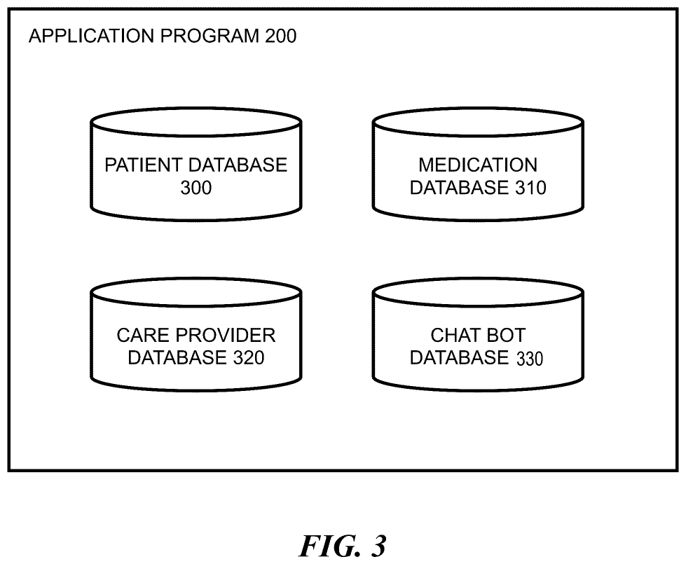

FIG. 3 illustrates a block diagram of the application program 200 which is in communication with a patient database 300, a medication database 310, a care provider database 320, and a chat bot database 330. The patient database 300 stores patient-associated information including the patient's medical history, condition, symptoms, and patient preferences. The medication database 310 stores medication-associated information such that an appropriate medication can be determined based on the systems analysis of the patient's condition. The medication database 310 may utilize a medical dataset to enable the determination, via the conversation engine, of the appropriate medication based on the patient's symptoms, medical history, and inferred condition. The care provider database 320 stores care provider (i.e., medical professional) information. The chat bot database 330 is in communication with the conversation engine to provide conversational language to the chat bot. This enables the chat bot to generate and transmit relevant and accurate response to inputs provided by the patient.

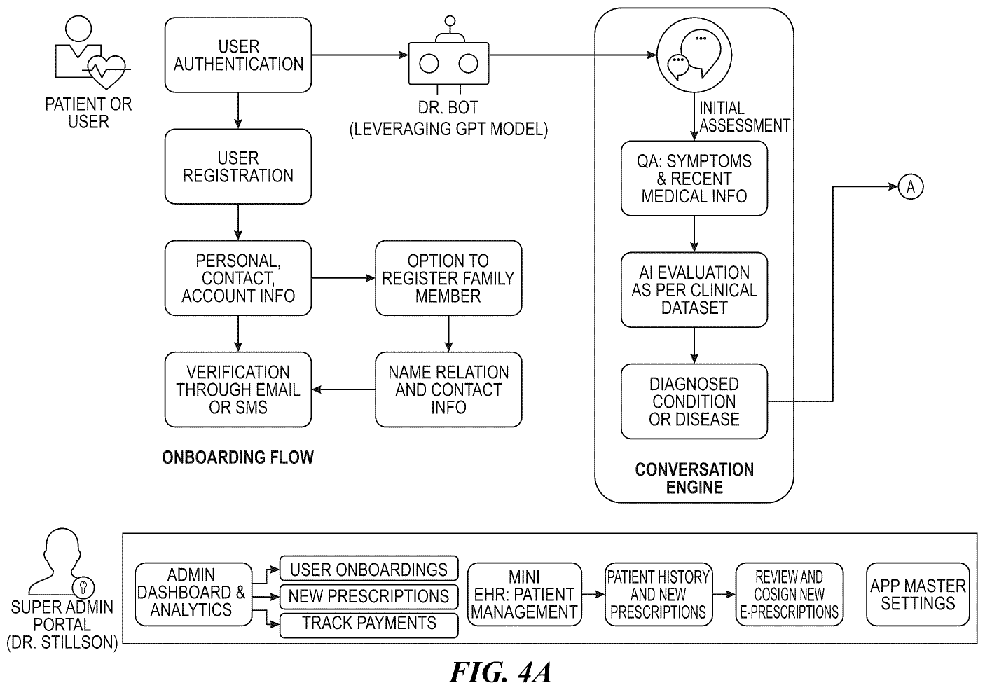

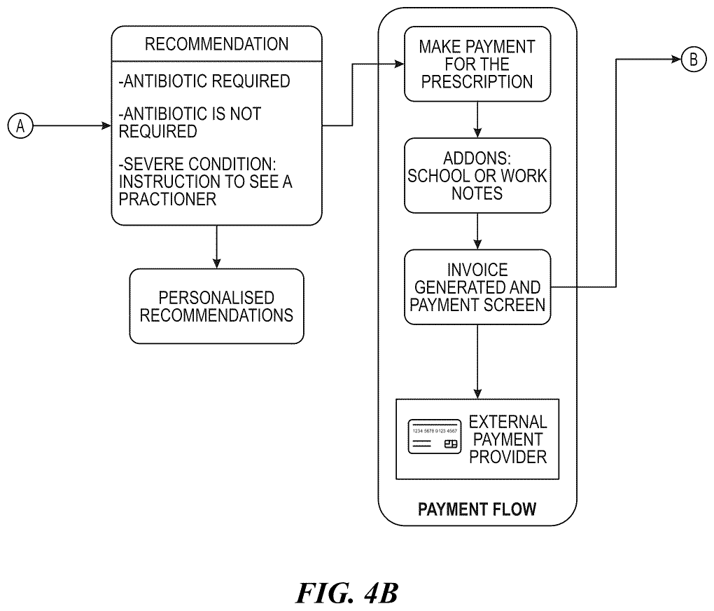

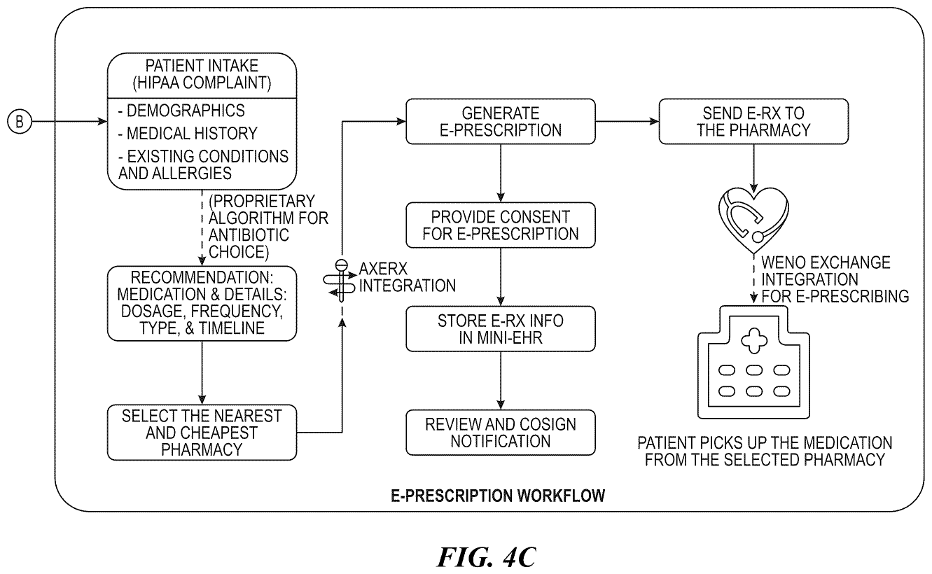

FIG. 4 illustrates a flowchart of a process for automating patient condition analysis and prescription generation. During an onboarding process, the patient (i.e., the user) is authenticated and registers with the application program. A user profile is created which includes the patient's personal information, contact information, account information, etc. Once registered, a verification email or SMS message may be transmitted to verify the patient's identity and credentials.

If the patient has already registered with the system, the patient may interact with a chat bot which utilizes artificial intelligence (AI) and machine learning (ML) to generate responses based on the patient's input. The chat bot utilizes a conversation engine which provides an initial assessment. The initial assessment is generated using questions which are transmitted to the patient. The conversation engine, through the use of AI and ML processes, evaluates the patient's condition using a clinical dataset, in order to diagnose the patient with a condition. Once the condition has been assessed and diagnosed, a recommendation for a medication is transmitted by the conversation engine. The recommendation may be that an antibiotic is required, that an antibiotic is not required, or that the condition is severe and further consultation with a physician is required in order to treat the patient's condition. If the recommendation is that an antibiotic is required to treat the patient's condition, a payment processing module requests payment from the patient and an invoice is generated. The specific medication which is recommended is then transmitted to the prescription generation module.

The prescription generation module may receive patient-associated information and the recommended medication to begin the generation of the e-prescription. The user may then select a pharmacy, or a pharmacy may be suggested based on the user's location. This allows the prescription generation module to generate an e-prescription which includes the patient's information, medication information, and pharmacy that will fill the prescription. A physician (or other medical professionals who can e-prescribe a medicine) may then review, verify, and sign the e-prescription. Once the AI-bot has reviewed, verified, and signed the e-prescription, the e-prescription is then transmitted to the selected pharmacy.

An administrative portal is provided which allows an administrative user (i.e., a medical professional) to view user onboardings, view prescriptions, track payments, and otherwise monitor the various functionalities of the system. This allows the administrator to manage each patient, view patient medical history, sign e-prescriptions, and otherwise interact with the system, patients and/or pharmacies.

In this disclosure, the various embodiments are described with reference to the flowchart illustrations and/or block diagrams of methods, apparatus (systems), and computer program products.

Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for automating patient condition analysis and prescription generation, the system comprising:
at least one user computing device in operable connection with a user network;
an application server in operable communication with the user network, the application server configured to host an application program for providing healthcare services and enabling an interaction between a healthcare provider using artificial intelligence and a patient, the application program having a user interface module for providing access to the application program via the at least one user computing device;
a conversation engine to analyze a plurality of patient symptoms and a plurality of patient medical information to evaluate the plurality of patient symptoms and the plurality of patient medical information, the conversation engine including an AI-enabled chat bot configured to transmit automated questions, receive patient-input communications via the user interface, and evaluate the patient symptoms and medical information using a clinical dataset of acute conditions stored in system databases;
wherein the conversation engine is further configured to identify a non-qualified condition, a qualified condition that allows for a prescription, and a high risk condition that necessitates a face-to-face medical evaluation, and to generate a recommended medication when a qualified condition is identified;
a comparator in operable communication with the conversation engine to compare the plurality of patient symptoms and the plurality of patient medical information to a clinical dataset to enable the determination of a medication from a medication dataset, the clinical dataset and medication dataset being stored in a medication database and accessed via a database engine;
a prescription generation module to associate the medication with the patient to generate an e-prescription, the e-prescription including patient identifiers and medication attributes and being formatted for electronic prescribing and digitally signed by the system;
a communication module to transmit the e-prescription to a pharmacy; and
a location module to determine a patient location and provides a list of pharmacies within a predefined radius for selection, the communication module securely transmits the e-prescription to the selected pharmacy and records the transmission, and an administrative portal exposes the generated e-prescription for asynchronous physician validation after transmission; and wherein a payment processing module processes patient payment and generates an invoice prior to transmission of the e-prescription.

2. The system of claim 1, further comprising a location module to determine the location of the patient and to provide a list of pharmacies within a predefined radius of the location of the patient.

3. The system of claim 1, further comprising a payment processing module to receive a payment from the patient and to distribute at least a portion of the payment to one or more recipients.

4. The system of claim 3, wherein the payment processing module is operable to generate an invoice associated with the e-prescription.

5. The system of claim 1, further comprising a patient database to store a plurality of patient information comprising: the plurality of patient symptoms, a patient medical history, and a plurality of patient identifying information.

6. The system of claim 1, further comprising a medication database to store the medication dataset, wherein the medication dataset is comprised of a plurality of antibiotics.

7. The system of claim 6, wherein the medication database stores a plurality of medications effective in treating one or more of a plurality of acute conditions comprising the clinical dataset.

8. The system of claim 1, wherein the conversation engine includes an AI-enabled chat bot operable to perform the following:
transmit automated communications to the patient;
receive a plurality of patient-input communications; and
analyze the plurality of patient-input communication to recommend the medication to the patient to e-prescribe the medication for the patient to the selected pharmacy in a licensed prescriber's name; and
to identify a patient condition and determine if it is a qualified condition for a prescription and then recommend the medication to the patient based on the product algorithm.

9. The system of claim 1, further comprising an administrative portal being accessible via the healthcare provider.

10. The system of claim 9, wherein the administrative portal is operable to permit the healthcare provider to review and to validate the e-prescription.

11. A system for automating patient analysis of acute medical conditions and prescription generation, the system comprising:
at least one user computing device in operable connection with a user network;
an application server in operable communication with the user network, the application server configured to host an application program for providing healthcare services and enabling an interaction between a healthcare provider and a patient, the application program having a user interface module for providing access to the application program via the at least one user computing device;
a conversation engine operating an AI-enabled chat bot to analyze a plurality of patient symptoms and a plurality of patient medical information to evaluate the plurality of patient symptoms and the plurality of patient medical information, the conversation engine including an AI-enabled chat bot configured to transmit automated questions, receive patient-input communications via the user interface, and evaluate the patient symptoms and medical information using a clinical dataset of acute conditions stored in system databases;
wherein the conversation engine is further configured to identify a non-qualified condition, a qualified condition that allows for a prescription, and a high risk condition that necessitates a face-to-face medical evaluation, and to generate a recommended medication when a qualified condition is identified;
a comparator in operable communication with the conversation engine to compare the plurality of patient symptoms and the plurality of patient medical information to a clinical dataset to enable the determination of a medication from a medication dataset, the clinical dataset and medication dataset being stored in a medication database and accessed via a database engine;
a prescription generation module to associate the medication with the patient to generate an e-prescription, the e-prescription including patient identifiers and medication attributes and being formatted for electronic prescribing and digitally signed by the system;
an administrative portal being accessible via the healthcare provider, the administrative portal to permit the healthcare provide to review the e-prescription and to validate, via an e-signature, the e-prescription;
a communication module to transmit the validated e-prescription to a pharmacy having a pharmacy API in operable communication with the application server, the communication module to provide an automated process of delivering validated e-prescription to the pharmacy; and
a location module to determine a patient location and provides a list of pharmacies within a predefined radius for selection, the communication module securely transmits the e-prescription to the selected pharmacy and records the transmission, and an administrative portal exposes the generated e-prescription for asynchronous physician validation after transmission;
and wherein a payment processing module processes patient payment and generates an invoice prior to transmission of the e-prescription.

12. The system of claim 11, further comprising a location module to determine the location of the patient and to provide a list of pharmacies within a predefined radius of the location of the patient.

13. The system of claim 12, further comprising a payment processing module to receive a payment from the patient and to distribute at least a portion of the payment to one or more recipients.

14. The system of claim 13, wherein the payment processing module is operable to generate an invoice associated with the e-prescription, wherein the payment processing module is in operable communication with the pharmacy API.

15. The system of claim 14, further comprising a patient database to store a plurality of patient information comprising: the plurality of patient symptoms, a patient medical history, and a plurality of patient identifying information, wherein the patient database is a HIPAA compliant electronic health record.

16. The system of claim 15, further comprising a medication database to store the medication dataset, wherein the medication dataset is comprised of a plurality of antibiotics.

17. The system of claim 16, wherein the medication database stores a plurality of medications effective in treating one or more of a plurality of acute conditions comprising the clinical dataset.

18. The system of claim 17, wherein the AI-enabled chat bot operable to perform the following:
transmit automated communications to the patient;
receive a plurality of patient-input communications; and
analyze the plurality of patient-input communication to recommend the medication to the patient to e-prescribe the medication for the patient to the selected pharmacy in a licensed prescriber's name.

19. The system of claim 18, wherein the e-prescription is comprised of a recommended dosage and a dosage schedule.

20. A method for automating patient analysis and prescription generation, the method comprising the steps of:
analyzing, via a conversation engine, a plurality of patient symptoms and a plurality of patient medical information to evaluate the plurality of patient symptoms and the plurality of patient medical information, wherein the conversation engine includes an AI-enabled chat bot that transmits automated questions to the patient, receives patient-input communications via a user interface, and evaluates the patient symptoms and medical information using a clinical dataset of acute conditions stored in system databases and accessed via a database engine;
comparing, via the conversation engine in operable communication with a comparator, the plurality of patient symptoms and the plurality of patient medical information to a clinical dataset to enable the determination of a medication from a medication dataset, wherein the comparator compares the patient symptoms and medical information to the clinical dataset to identify a non-qualified condition, a qualified condition permitting a prescription, or a high-risk condition requiring face-to-face evaluation, and upon identifying a qualified condition the conversation engine generates a recommended medication from a medication dataset stored in a medication database;
associating, via a prescription generation module, the medication with the patient to generate an e-prescription, wherein the prescription generation module constructs the e-prescription with patient identifiers and medication attributes and formats the e-prescription for electronic prescribing;
selecting, via a location module, a pharmacy for fulfillment by determining a patient location and providing a list of pharmacies within a predefined radius for patient selection;
processing, via a payment processing module, patient payment and generating an invoice associated with the e-prescription prior to transmission;
transmitting, via a communication module, the e-prescription to a pharmacy, wherein the communication module securely transmits the e-prescription to the selected pharmacy and records the transmission; and
validating, via a healthcare provider accessing an administrative portal, the e-prescription.
